(19) United States
(12) Patent Application Publication
(10) Pub. No.: US 2025/0160835 A1
(43) Pub. Date: May 22, 2025

(54) A CATHETER SET FOR FORMING A FISTULA BETWEEN TWO BLOOD VESSELS

(71) Applicant: CLEARSTREAM TECHNOLOGIES LIMITED, nniscorthy, County Wexford (IE)

(72) Inventors: Jakob Wells, Enniscorthy, County Wexford (IE); Michael Whelan, Enniscorthy, County Wexford (IE); John O'Shea, Enniscorthy, County Wexford (IE)

(21) Appl. No.: 18/874,995

(22) PCT Filed: Jun. 13, 2022
(86) PCT No.: PCT/EP2022/065993

§ 371 Date: Dec. 13, 2024

(51) Int. Cl. A61B 17/11; A61B 18/00; A61B 18/14

(52) U.S. Cl. CPC A61B 17/11; A61B 18/1492; A61B 2017/1107; A61B 2018/00404; A61B 2018/1407; A61B 2018/144

(57) ABSTRACT

The present invention relates to a catheter for forming a fistula between two vessels comprising a catheter body having a longitudinal axis; a first electrode extending radially from the catheter body for contacting a vessel wall and forming the fistula; and a second electrode disposed on the catheter body for ablating a valve within a vessel.

### TECHNICAL FIELD

The present disclosure relates to a catheter set for forming a fistula between two adjacent blood vessels and a method of creating arterial bloodflow in a vein.

### BACKGROUND OF THE INVENTION

The accumulation of atherosclerotic plaque in blood vessel walls can often result in blood flow blockages, which can cut or reduce arterial circulation to limbs and lead to peripheral artery disease. Resulting conditions can include infection, tissue death and in some cases total limb necrosis.

Alleviating peripheral arterial diseases can make use of minimally invasive treatments including angioplasty and atherectomy, whereby catheters are inserted to respectively widen the blood vessel with a balloon or remove plaque from the vessel wall. Other types of procedures include endovascular bypass procedures or deep vein arterialization (DVA) procedures. Both of these procedures require the creation of a fistula between a vein and artery and the destruction of venous valves to allow retrograde blood flow in the vein. For an endovascular bypass procedure, a vascular bypass graft is placed from a location proximal to the blockage to a location distal to the blockage in order to provide an unobstructed path for the blood to circumvent the diseased area. For a deep vein arterialization procedure, a fistula is created between a peripheral artery and vein with the intent of “arterializing” the veins. Arterial blood flows through the veins and bypasses the blockage in the artery to thereby provide adequate blood flow to the target tissue.

Current devices used to perform endovascular bypass and DVA procedures make use of multiple catheters, each configured to perform one specific step of the procedure, such as valve destruction, or fistula creation. The use of multiple catheters makes the procedure more complex and time-consuming, more particularly so during emergency procedures.

Moreover, the destruction of venous valves during these procedures typically relies on mechanical cutting elements. In some cases, these mechanical cutting elements are not effective at destroying the venous valves and may be complex to allow the vein to be cut without damaging the vessel wall.

For example, US 2017/0202616 A1 and US 2014/0142677 A1 disclose systems for forming an arteriovenous fistula. US 2016/0100855 A1 discloses a catheter for destroying venous valves.

There is hence a need in the art for a catheter system which can simplify and reduce the number of steps in an endovascular bypass or DVA procedure and thereby reduce the time taken to perform these procedures.

There is further a need in the art for a catheter system which can provide a simple, safe and effective way of destroying venous valves during endovascular bypass, DVA or similar procedures.

### SUMMARY

According to a first aspect of the disclosure, there is provided a catheter for forming a fistula between two vessels. The catheter comprises a catheter body having a longitudinal axis, a first electrode extending radially from the catheter body configured to contact a vessel wall and forming the fistula, and a second electrode disposed on the catheter body configured to ablate a valve within a vessel.

In some embodiments, the this may result in a catheter system which can simplify and reduce the number of steps and the number of catheter insertions during procedures such as deep vein arterializations or endovascular bypass procedures.

Throughout this disclosure, the term ‘ablation’ of a valve refers to the destruction or incapacitation of a valve in the vein, enabling retrograde blood flow in the vein.

Throughout this disclosure, the term of ‘fistula’ refers to a passageway or connection, for example between an artery and a vein.

The second electrode may be disposed distally of the first electrode.

In some embodiments this may allow the catheter to better destroy venous valves when advancing to or withdrawing from the treatment site.

The second electrode may be disposed circumferentially around the catheter body.

In some embodiments this may result in circumferential contact of the electrode with the valve and more effective destruction of the valve.

The second electrode may be ring-shaped. In some embodiments this may result in circumferential contact of the electrode with the valve and more effective destruction of the valve.

The second electrode may comprise two or more portions which are disposed circumferentially around the catheter body. In some embodiments this may result in effective destruction of a valve.

The two portions of the second electrode may be disposed on opposite sides of the catheter body. In some embodiments this may result both cusps of a valve being contacted by an electrode portion and therefore more effective destruction of the valve.

The second electrode may extend radially from the catheter body. In some embodiments this may allow the electrode to better contact the valve and result in better valve destruction.

The catheter body may further comprise protrusions either side of the second electrode for holding the second electrode in place. In some embodiments this may result in a secure attachment of the second electrode with the catheter body and added stability when destroying a valve.

The first electrode may have a radially expanded configuration and a contracted configuration. In some embodiments this may allow the first electrode to be advanced through the vessel anatomy in the contracted configuration having a low profile and expanded to create a fistula, resulting in a more efficient fistula-creating process.

Throughout this disclosure, the term ‘radially expanded configuration’ of the electrode is used to denote a configuration in which the electrode extends radially further from the housing than in a ‘radially contracted configuration’.

The first electrode may further comprise a distal portion, a proximal portion and an intermediate portion therebetween for contacting a vessel wall and forming the fistula.

The intermediate portion may have a convex shape. In some embodiments this may result in more effective fistula creation.

The catheter body may further comprise a housing. The first electrode may at least be partially disposed within the housing. In some embodiments this may better protect the first electrode.

The housing may be at least partly made from a ceramic material. In some embodiments this may allow the housing to better withstand the heat and plasma generated by the electrode.

The first electrode may further comprise a leaf spring. In some embodiments, this may allow the first electrode to more easily move between the expanded configuration and the contracted configuration.

Throughout this disclosure, the term ‘leaf spring’ is used to refer to a flexible curved strip of material which can be bent but will regain its original shape when released.

The first electrode may further comprise a ribbon wire. In some embodiments this may result in more effective fistula creation.

The first electrode and/or the second electrode may be configured to carry a radiofrequency current. In some embodiments this may enable efficient valve ablation or fistula creation.

The catheter may further comprise one or more magnets. In some embodiments this may assist in aligning of the first electrode with a backstop.

The catheter may further comprise a proximal set of magnets, disposed proximally of the first electrode. In some embodiments this may assist in aligning of the first electrode with a backstop.

The catheter may further comprise a distal set of magnets, disposed distally of the first electrode. In some embodiments this may assist in aligning of the first electrode with a backstop.

The second electrode may be disposed distally of the distal set of magnets. In some embodiments this may allow the catheter to better destroy venous valves when advancing to or withdrawing from the treatment site.

The catheter may further comprise a rapid exchange tip at the distal end of the catheter body. In some embodiments this may enable faster exchange of the catheter.

The catheter body may further comprise a braided catheter portion. In some embodiments this may assist in stabilising the catheter along the blood vessel.

The catheter may further comprise a first electrode connecting element connected to the first electrode and extending along the length of the catheter body. In some embodiments this may assist in powering the first electrode independently.

The catheter may further comprise a second electrode connecting element connected to the second electrode and extending along the length of the catheter body. In some embodiments this may assist in powering the second electrode independently.

According to a second aspect of the disclosure, there is provided a system for forming a fistula between two vessels comprising a first catheter and a handle attached to the proximal end of the first catheter. In some embodiments, this may facilitate operation of the first catheter.

The handle may further comprise a toggle switch.

The toggle switch may further be configured to switch between providing power to the first electrode or the second electrode. In some embodiments, this may enable independent and separate operation of each electrode.

The system may further comprise a radiofrequency generator for supplying radiofrequency power to the first and second electrode. In some embodiments, this may allow the fistula forming process and the valve destruction process to be powered with the same power supply, resulting in a more efficient procedure.

The system may further comprise a second catheter having a backstop. In some embodiments this may result in better fistula formation.

The backstop may be made from a non-conductive material.

The backstop may be made, at least partly, from a ceramic material. In some embodiments, this may allow the backstop to better withstand the heat and plasma generated by the first electrode.

The backstop may further comprise a recessed portion which has a complimentary shape to the first electrode. In some embodiments, this results in more effective fistula formation.

The recessed portion may be of a concave shape.

The second catheter may further comprise a proximal set of magnets, disposed proximally of the backstop. In some embodiments this may assist in aligning the first electrode with the backstop.

The second catheter may further comprise a distal set of magnets, disposed distally of the backstop. In some embodiments this may assist in aligning the first electrode with the backstop.

According to a third aspect of the disclosure, there is provided a method of creating arterial bloodflow in a vein, using a catheter having a catheter body with a first electrode and a second electrode. The method comprises inserting the catheter into a vein through an access site. The method comprises advancing the catheter to a treatment site where a fistula is to be formed. Upon encountering a valve, the method comprises supplying RF energy to the second electrode to destroy said valve. Upon arriving at a treatment site where the fistula is to be formed, the method comprises supplying RF energy to the first electrode to form the fistula.

In some embodiments this may provide a simple method to reduce the number of steps for DVA or endovascular bypass procedures.

### BRIEF DESCRIPTION OF THE DRAWINGS

For a more complete understanding of the present disclosure, reference is now made, by way of example only, to the accompanying drawings. These drawings are used to illustrate only typical embodiments of this disclosure and are not to be considered limiting on its scope. The figures are not necessarily to scale and certain features and certain views of the figures may be shown exaggerated in scale or in schematic in the interest of clarity and conciseness.

FIG. 1A is a schematic representation of a system with a first catheter and a handle attached to the proximal end of the first catheter, with a fistula-creating electrode in a radially contracted configuration, according to the present disclosure;

FIG. 1B is a schematic representation of the system of FIG. 1A, with the fistula-creating electrode in the radially expanded configuration, according to the present disclosure;

FIG. 2 is a schematic representation of a second catheter comprising a backstop, according to the present disclosure;

FIG. 3A is a schematic representation of a system comprising the first catheter of FIGS. 1A and B and the second catheter of FIG. 2, positioned in a vein and artery respectively;

FIG. 3B is a schematic representation of a system comprising the first catheter of FIGS. 1A and B and the second catheter of FIG. 2, during a valve destruction process;

FIG. 3C is a schematic representation of a system comprising the first catheter of FIGS. 1A and B and the second catheter of FIG. 2, during a fistula creation process;

FIG. 3D is a schematic representation of blood flow through the artery and vein after the valve destruction and fistula creation process;

FIG. 4 is a schematic representation of an alternative embodiment of a first catheter according to the present disclosure; and

FIG. 5 is a schematic representation of an alternative embodiment of a first catheter according to the present disclosure.

### DETAILED DESCRIPTION

The embodiments described herein are provided as exemplary and non-limiting embodiments of the present disclosure. The invention is defined by the appended claims.

Directional terms as used herein—for example up, down, right, left, front, back, top, bottom, distal, and proximal—are made only with reference to the figures as drawn and are not intended to imply absolute orientation.

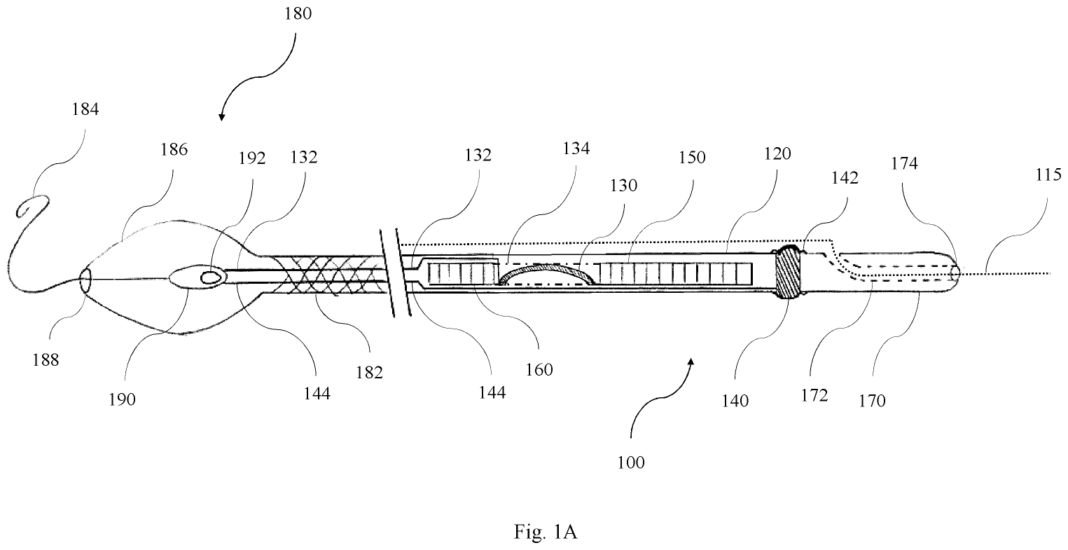

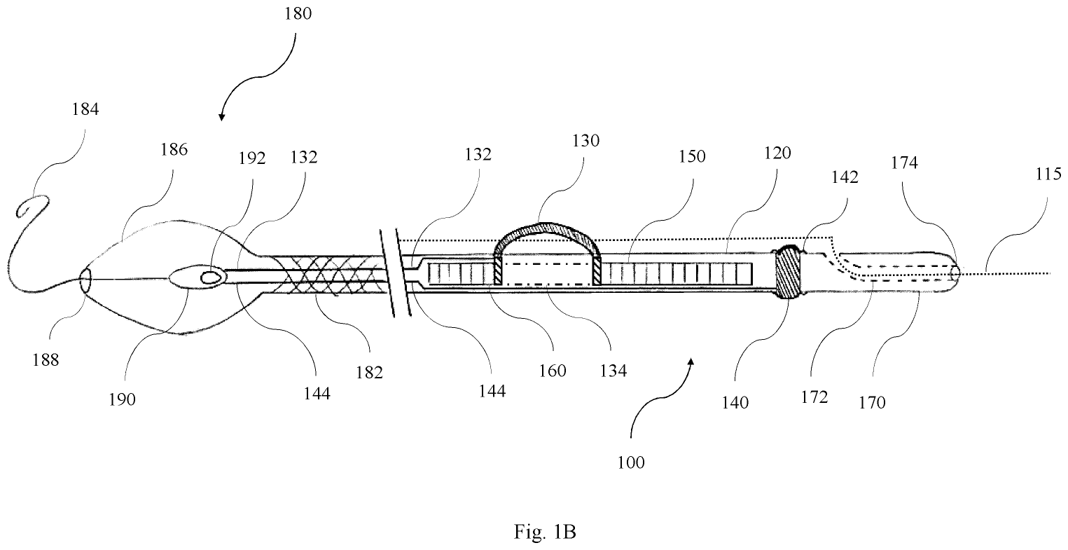

With reference to FIGS. 1A and 1B, there is illustrated a first catheter 100 and a handle 180 attached to the proximal end of the first catheter 100. The first catheter 100 comprises a catheter body 120 which may have a tubular shape.

The catheter body 120 comprises a first electrode 130 which may extend radially from the catheter body 120 for contacting a vessel wall, such as a wall of a vein, and forming a fistula. The first electrode 130 may be in the form of a ribbon wire and have an arc shape which is convex with respect to the longitudinal axis of the catheter body 120. The first electrode 130 may be made from a number of suitable materials such as tungsten, molybdenum, niobium, tantalum, rhenium, or combinations and alloys thereof.

The first electrode 130 is positioned at least partially within the electrode housing 134. The electrode housing 134 may be made from a non-conductive material, such as a ceramic material, which may allow the electrode housing 134 to better withstand the heat from the plasma generated by the first electrode 130. The first electrode 130 may have a radially contracted configuration shown in FIG. 1A and a radially expanded configuration shown in FIG. 1B.

In the radially expanded configuration of FIG. 1B, the first electrode 130 may extend radially further away from the electrode housing 134 than in the radially contracted configuration of FIG. 1A. In the radially contracted configuration shown in FIG. 1A, the electrode 130 may be fully disposed in the housing 134, or it may extend from the housing 134 by a small distance. The first electrode 130 can be in the form of a leaf spring which may allow the first electrode 130 to bend and thereby effectively move between the radially contracted and radially expanded configurations.

There are a number of different ways in which the first electrode 130 may be moved between the radially contracted configuration and the radially expanded configuration. For example, the distal end of the first electrode 130 may be free to move within the electrode housing 134. The first electrode 130 can then be moved from the radially expanded configuration (see FIG. 1B) to the radially contracted configuration (see FIG. 1A) by pushing down on the first electrode 130, for example, through the use of a sheath (not shown). When the sheath is removed, the first electrode 130 will regain its original shape and expand from the radially contracted configuration to the radially expanded configuration.

In another example, the distal end of the first electrode 130 can be fixed relative to the electrode housing 134. The proximal end of the first electrode 130, which may be connected to a connecting element 132, can be longitudinally moveable relative to the electrode housing 134. A user may then push or pull the proximal end of the first electrode 130 via a connecting element 132 in order to move the first electrode 130 between the radially contracted configuration and the radially expanded configuration. For example, a user may push the connecting element 132 and the proximal end of first electrode 130 in order to move the first electrode 130 from the radially contracted configuration to the radially expanded configuration. Similarly, a user may pull the connecting element 132 and the proximal end of the first electrode 130 in order to move the first electrode 130 from the radially expanded configuration to the radially contracted configuration.

These configurations of the first electrode 130 may be advantageous for a more efficient fistula-creating process, in that the first electrode 130 can be advanced through the vessel anatomy in the radially contracted configuration having a low profile, and subsequently expanded to create a fistula in the radially expanded configuration.

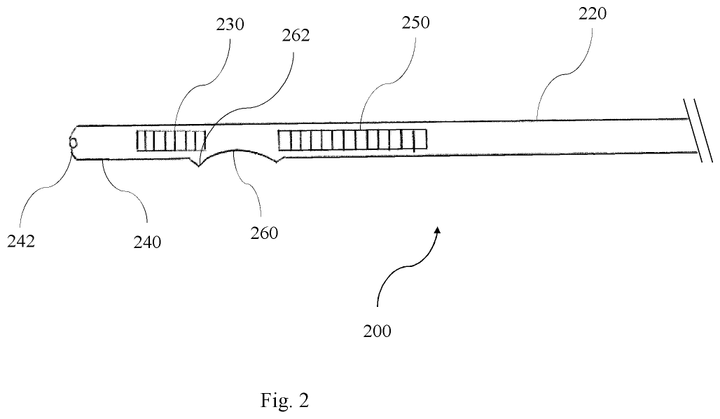

The catheter body 120 may further comprise a proximal set of magnets 160 disposed proximally of the first electrode 130. The catheter body 120 may also further comprise a distal set of magnets 150 disposed distally of the first electrode 130. The magnets may be used to assist in aligning the first electrode 130 with a backstop 260 (shown in FIG. 2). In some configurations, a single set of magnets may suffice. The number of magnets disposed within the set of magnets 150 and 160 is variable and any suitable number of magnets may be used.

The first catheter 100 may also comprise a rapid exchange tip 170 at the distal end of the catheter body 120. The rapid exchange tip 170 may comprise a guidewire lumen 172 for accommodating a guidewire 115 which ends in a distal opening 174. The rapid exchange tip 170 may enable a faster exchange of the first catheter 100.

The first catheter 100 further comprises a second electrode 140 for ablating a valve within a vessel, such as a vein V. The second electrode 140 may be positioned distal of the first electrode 130 and proximal of the rapid exchange tip 170 and may extend circumferentially around the catheter body 120. The second electrode 140 may be made from a number of suitable materials such as nitinol, tungsten, molybdenum, niobium, tantalum, rhenium, or combinations and alloys thereof. As shown in FIGS. 1A and B, the second electrode 140 may be ring-shaped. This may allow the second electrode 140 to contact a circumferential section of the valve to therefore render the valve destruction more effective. Protrusions 142 on the surface of the catheter body 120 may be disposed either side of the second electrode 140 for holding the second electrode 140 in place. Protrusions 142 provide a secure attachment of the second electrode 140 with the catheter body 120 and added stability when destroying a valve.

The first electrode 130 may be connected at its proximal end to the first electrode connecting element 132 and the second electrode 140 may be connected at its proximal end to a second electrode connecting element 144. The first and second electrode connecting elements 132 and 144 may extend along the length of the catheter body 120. First and second electrode connecting elements 132 and 144 may connect the first and second electrodes 130 and 140 to a source of RF energy, which may be in the form of an ESU pencil (examples of which include a Bovie® Pen Generators), such that RF energy can be supplied to each of the first and second electrodes 130, 140 independently. First and second electrode connecting elements 132 and 144 may be conductive and configured to carry an RF current, so as to allow supply of RF energy to the first and second electrodes 130 and 140 respectively.

A handle 180 may be disposed at a proximal end of the first catheter 100. Handle 180 may comprise a braided catheter end 182 which is connected to the distal end of catheter body 120. The braided catheter end 182 may facilitate operation of the first catheter 100 by providing grip, thus stabilising the first catheter 100. First and second electrode connecting elements 132 and 144 may extend within the catheter body 120, and inside braided catheter end 182 up to a toggle switch 190. Toggle switch 190 may be positioned on the grip handle 186 and may comprise a toggle switch button 192. The toggle switch 190 is configured to alternate between providing power to the first and the second electrode 130, 140, by moving the toggle switch button 192 between a first and second configuration. Grip handle 186 may have a bulbous or otherwise conveniently manoeuvrable shape and is connected at a distal end to the braided catheter end. The proximal end of grip handle 186 may include an aperture 188 through which a third connecting element 184 passes. Third connecting element 184 is connected directly at a distal end to toggle switch 190, and indirectly to electrode connecting elements 132 and 144 via the toggle switch 190. The third connecting element 184 may be connected proximally to the source of RF energy, which may be in the form of a generator or ESU pencil (not illustrated).

FIG. 2 shows a second catheter 200 which may form a system together with first catheter 100 to form a fistula between two blood vessels. Second catheter 200 may comprise a catheter body 220 and a backstop 260 having a recessed portion, which is concave with respect to the catheter body 220. Protuberances 262 protrude radially outward from the catheter body 220 and assist in creating a concave geometry. The concave geometry may be complementary to the shape of the first electrode 130 and may help to induce a deformation of the vessel wall. The second catheter 200, the catheter body 220 and the backstop 260 may be made from a non-conductive material, such as a ceramic material, such that the material can withstand the heat from the plasma generated by the first electrode 130.

The catheter body 220 may further comprise a proximal set of magnets 230 disposed proximally of the backstop 260. The catheter body 220 may further comprise a distal set of magnets 250 disposed distally of the backstop 260. The sets of magnets 235, 250 may be used for aligning the backstop 260 of the second catheter 200 with the first electrode 130 of the first catheter 100. In some configurations a single set of magnets may suffice. The number of magnets disposed within each set of magnets 250 and 230 is variable.

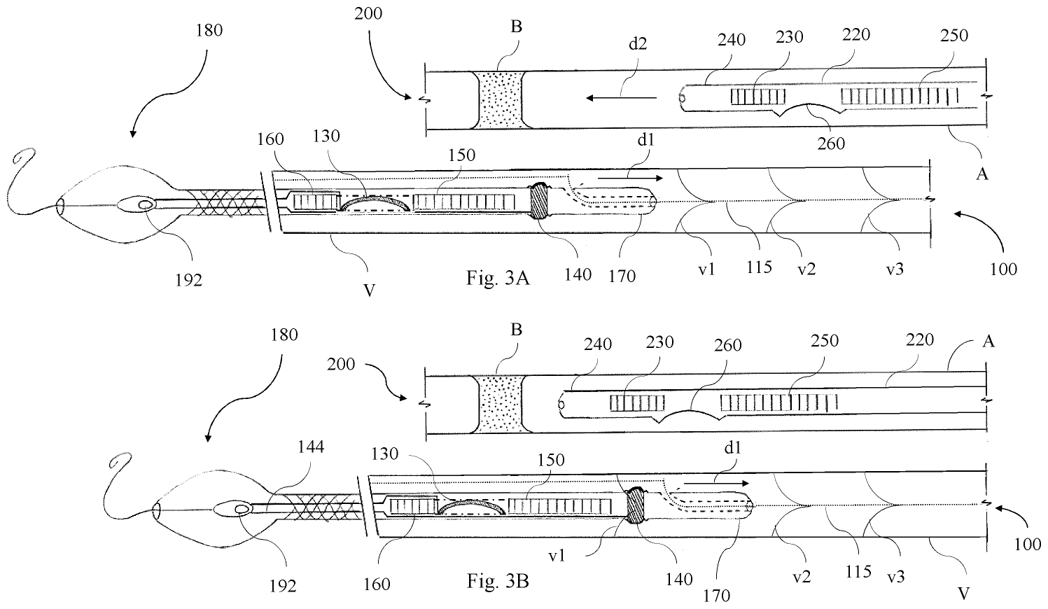

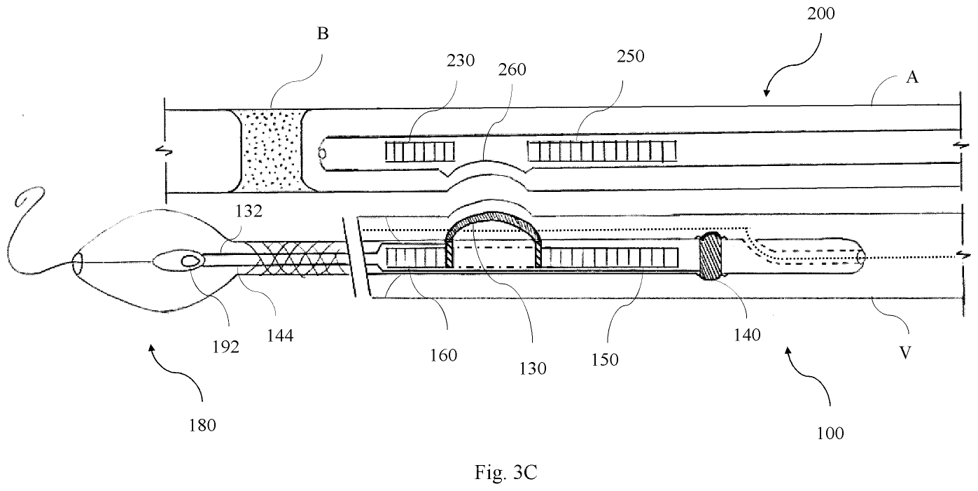

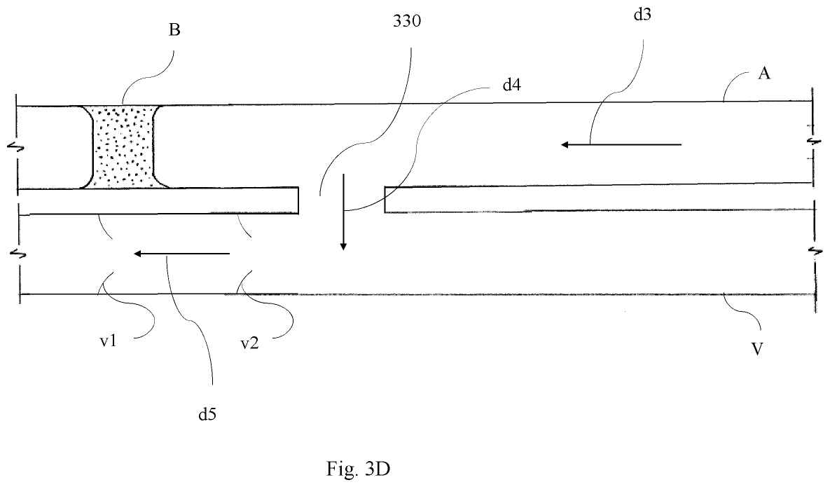

FIGS. 3A-3D illustrate a method of using the first catheter 100 and the second catheter 200 to destroy the valves in a vein V and form a fistula between the vein V and an artery A to allow blood to flow past a blockage B in the artery.

FIG. 3A shows a cross-sectional side view of the first catheter 100 disposed in a vein V in a first position and the second catheter 200 disposed in an artery A in a first position, the first and second catheters 100, 200 being advanced to a treatment site where a fistula is to be formed.

Firstly, the first catheter 100 is introduced into a vein V through an access site and advanced along guidewire 115 towards the treatment site in a first direction d1. The first direction d1 may be a proximal direction. To facilitate introduction into and advancement of the first catheter 100 along vein V, the first electrode 130 of the first catheter 100 may be in the radially contracted configuration. The first catheter 100 may encounter a number of venous valves such as v1, v2 or v3 shown in FIG. 3A, which block retrograde flow of blood through the vein. In order to successfully perform a deep vein arterialization procedure or an endovascular bypass procedure, these valves must be rendered incompetent.

The second catheter 200 is introduced into an artery A through a second access site and advanced towards the treatment site in second direction d2. The second direction d2 may be a distal direction. In some embodiments, a second guidewire (not shown) may be used to guide the second catheter 200 to the treatment site. The first catheter 100 and/or the second catheter 200 may also be introduced into the vessel and advanced to the treatment site inside a sheath (not shown) which may protect the catheters.

FIG. 3B shows a cross-sectional side view of the catheter system where the first catheter 100 has been advanced to a second position. The second catheter 200 is shown as being positioned at the treatment site. In order to destroy a venous valve, such as valve v1, and render it incompetent, the first catheter 100 is positioned such that the second electrode 140 comes into contact with the venous valve v1.

The user may then press toggle switch button 192 on handle 180 to ensure that the toggle switch 190 is set to the second configuration where the RF energy is routed to the second electrode 140. The user may additionally ensure that the settings on the generator (not illustrated) are set to a valve destruction setting, where the RF power supplied to the second electrode 140 may be lower and the activation time may be shorter than for a fistula-creation setting for the first electrode 130. To prevent the second electrode 140 from activating on an incorrect setting the user may, for example, manually confirm the generator settings before activating the second electrode 140 or this may be prevented by means of at least one smart plug (not illustrated) connected to the toggle switch button 192 on handle 180. The smart plug may detect a change in resistance in the circuitry and ensure, for example, that the first electrode 130 and second electrode 140 are only activated on the correct respective settings.

Thereafter, a radiofrequency (RF) current may be supplied to the second electrode 140 through second electrode connecting element 144, which causes the second electrode 140 to heat up and generate a plasma. The plasma causes rapid dissociation of the molecular bonds in the organic compounds and allows the second electrode 140 to cut through the venous valve v1. The valve v1 is damaged and thus rendered incompetent, such that it can no longer prevent retrograde blood flow in the vein. The RF generator may provide an array of different cut modes, which can be selected to destroy the venous valve v1. The first catheter 100 may then be advanced until the second electrode comes into contact with the next valve v2 and the process may be repeated. The process may be repeated for any number of valves necessary for the treatment procedure.

Once all of the necessary valves are destroyed, the first and second catheter 100, 200 are advanced to the treatment site, where the fistula is to be formed. FIG. 3C shows a cross-sectional side view of the catheter system with the first catheter 100 in a third position inside the vein V at the treatment site. The second catheter 200 is also positioned at the treatment site in the artery A. The first electrode 130 is moved from the radially contracted configuration to the radially expanded configuration. In the radially expanded configuration, the first electrode 130 extends from the electrode housing 134 and comes into contact with the venous wall. The proximal set of magnets 160 of the first catheter 100 may be aligned with the distal set of magnets 230 of the second catheter 200. The distal set of magnets 150 of the first catheter 100 may be aligned with the proximal set of magnets 250 of the second catheter 200. This results in the first convex-shaped electrode 130 becoming aligned with the complimentary concave-shaped backstop 260. The magnets may further have the effect of reducing the distance between the vein V and artery A.

The user may subsequently press toggle switch button 192 on handle 180 to ensure that the toggle switch 190 is set to the first configuration such that the RF energy is routed to the first electrode 130. The user may additionally ensure that the settings on the generator (not illustrated) are set to the fistula-creation setting, where the RF power supplied to the first electrode 130 may be higher and the activation time may be longer than for the valve destruction setting. The first electrode 130 can be prevented from activating on an incorrect setting, for example, by manually confirming the generator settings before activation or through the use of a smart plug, as explained above. As an additional safety mechanism, the first electrode 130 may not be activated unless a number of conditions are met, for example, that the first electrode 130 is fully aligned with the backstop 260.

Thereafter, a radiofrequency (RF) current may be supplied to the electrode 130 through first electrode connecting element 132, which causes the first electrode 130 to heat up and generate a plasma. The plasma causes rapid dissociation of the molecular bonds in the organic compounds and allows the first electrode 130 to cut through the walls of the vein V and artery A until it reaches the backstop 260, thereby creating a fistula 330 connecting vein V to artery A. The first and second catheters 100, 200 may then be withdrawn from the vein V and artery A, respectively.

FIG. 3D is a cross-sectional view of the vein V and artery A after the first and second catheter 100, 200 have been withdrawn. FIG. 3D shows the fistula 330 which has been formed between the vein V and artery A, proximal of the blockage B. FIG. 3D also shows the venous valves v1 and v2 which have been rendered incompetent. Blood may therefore flow in the direction of the arrows d3, d4 and d5, from the artery A through the fistula 330, into the vein V, and in a retrograde direction through vein V.

When performing a deep vein arterialization (DVA) procedure, a stent may be placed within the fistula 330 to stabilise the fistula. When performing an endovascular bypass procedure, a second fistula may be formed distally of the blockage B in a similar manner as explained with respect to FIG. 3C above. A stent graft may then be placed through the first and second fistulas via the vein V, such that the blood flow can circumvent the blockage B.

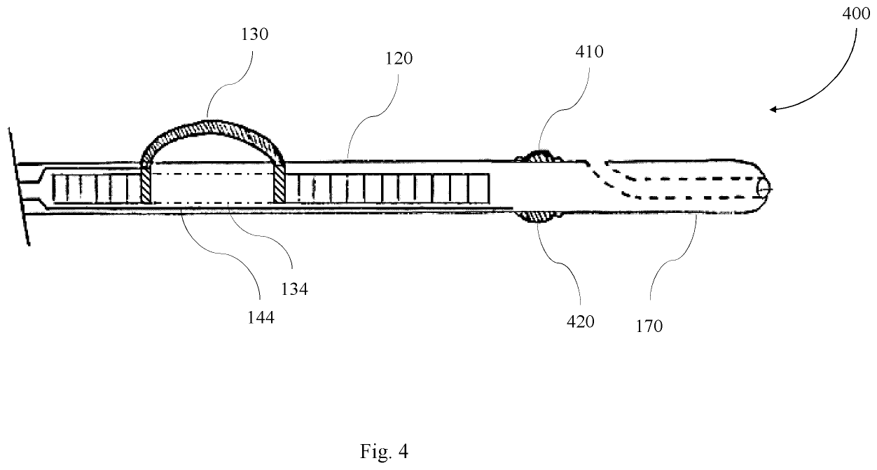

FIG. 4 shows an alternative embodiment of a first catheter 400. Throughout this disclosure, the same reference numerals are used to refer to features which are identical across different embodiments. The first catheter 400 is similar to the first catheter 100 of FIG. 1 in that it comprises catheter body 120, first electrode 130, electrode housing 134 and a rapid exchange tip 170. However, the first catheter 400 differs in that the second electrode comprises first and second electrode portions 410 and 420 disposed on opposite sides of the catheter body 120. The presence of two portions 410, 420 of the second electrode may result in both cusps of a valve being contacted by an electrode portion, and therefore effective destruction of the valve. The two electrode portions 410, 420 may also be independently activated to more specifically target sections of a valve.

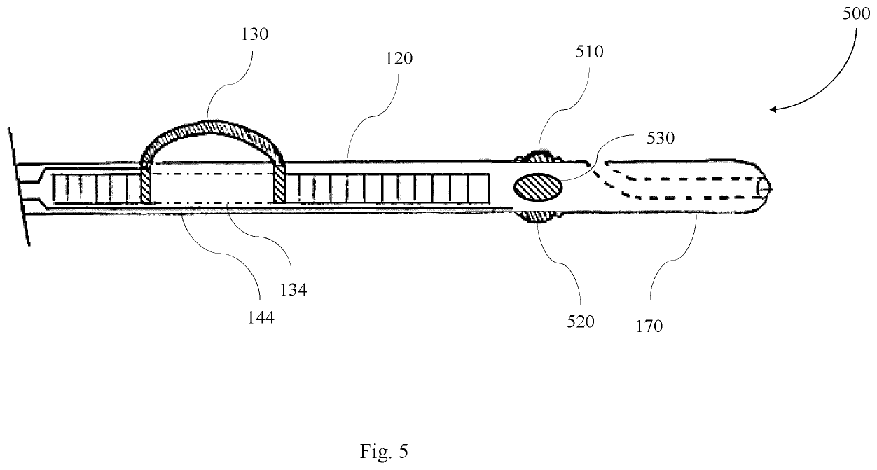

FIG. 5 shows another alternative embodiment of a first catheter 500 comprising a catheter body 120, first electrode 130, electrode housing 134 and rapid exchange tip 170. The first catheter 500 comprises a second electrode having a four electrode portions (only three electrode portions 510, 520 and 530 are shown) disposed circumferentially around the catheter body. However, the first catheter 500, may have any suitable number of electrode portions, for example, in the range of 2 to 8, preferably in the range of 4 to 6 electrode portions. Each of the plurality of electrode portions may be individually activated to more specifically target sections of a valve.

Thus, the present invention provides a device, system and method which can simplify and reduce the number of steps in an endovascular bypass or DVA procedure and thereby reduce the time taken to perform these procedures. The catheter system described herein additionally provides a simple, safe and effective way of destroying venous valves during endovascular bypass, DVA or similar procedures.

Various modifications will be apparent to those skilled in the art.

For example, the second electrode 140 may be positioned proximally of the first electrode 130. The second electrode 140 is not limited to being ring-shaped or circumferentially disposed around the catheter body 120. The second electrode 140 may have any suitable shape for destroying valve.

The second electrode 140 may be secured to the catheter body 120 via means other than the use of protrusions 142. For example, the second electrode 140 may be fixed directly to the catheter body 120 via an adhesive.

The first electrode 130 is not limited to having a convex shape but may take any other suitable shape such as a triangular or rectangular shape. The first electrode 130 is further not limited to being a ribbon wire but may be a wire having any other suitable shape, such as a circular, oval or square wire.

The first electrode 130 and the second electrode 140 may be made from any suitable material which can conduct an electric current.

The first electrode 130 may not have a radially contracted and a radially expanded configuration. Rather, the first electrode may only have one constant configuration.

The first catheter 100 may not comprise a rapid exchange tip 170.

The first catheter 100 may not be connected to a handle 180.

The backstop 260 of the second catheter 200 is not limited to having a concave portion and is not limited to having a shape that is complimentary to the first electrode 130. The backstop 260 may have any suitable shape, such as a rectangular shape, for example.

The electrode housing 134 and the backstop 260 are not limited to being made from ceramic materials. They can be made from any suitable materials which can withstand the heat and plasma generated by the first electrode 130.

The electrodes may be powered by means other than a generator and other than with RF energy.

The first and second catheters 100, 20 may not comprise any magnets. The alignment of the first and second catheters 100, 200 may be achieved through other means such as by fluoroscopy or with sensors embedded in the catheter shaft, for example.

The handle 180 may not comprise a braided catheter portion 182. The handle 180 further may not comprise a toggle switch 190. Another mechanism may be used to activate the first and second electrodes 130, 140, for example two switches or a digital screen.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter for forming a fistula between two vessels comprising:
a catheter body having a longitudinal axis;
a first electrode extending radially from the catheter body configured to contact a vessel wall and form the fistula; and
a second electrode disposed on the catheter body configured to ablate a valve within a vessel.

2. The catheter of claim 1, wherein the second electrode is disposed distally of the first electrode.

3. The catheter of claim 1, wherein the second electrode is disposed circumferentially around the catheter body.

4. The catheter of claim 1, wherein the second electrode is ring-shaped.

5. The catheter of claim 1, wherein the second electrode comprises two or more portions which are disposed circumferentially around the catheter body.

6. The catheter of claim 5, wherein the second electrode comprises two portions which are disposed on opposite sides of the catheter body.

7. The catheter of claim 1, wherein the second electrode extends radially from the catheter body.

8. The catheter of claim 1, wherein the catheter body comprises protrusions either side of the second electrode for holding the second electrode in place.

9. The catheter of claim 1, wherein the first electrode has a radially expanded configuration and a radially contracted configuration.

10. The catheter of claim 1, wherein the first electrode comprises a distal portion, a proximal portion and an intermediate portion therebetween for contacting a vessel wall and forming the fistula.

11. (canceled)

12. The catheter of claim 1, wherein the catheter body comprises a housing and the first electrode is at least partially disposed within the housing.

13. The catheter of claim 12, wherein the housing is at least partly made from a ceramic material.

14. The catheter of claim 1, wherein the first electrode comprises a leaf spring, or a ribbon wire.

15. (canceled)

16. The catheter of claim 1, wherein the first electrode and/or the second electrode are configured carrying a radiofrequency current.

17. (canceled)

18. The catheter of claim 1, further comprising a proximal set of magnets, disposed proximally of the first electrode.

19. The catheter of claim 1, further comprising a distal set of magnets, disposed distally of the first electrode.

20. The catheter of claim 16, wherein the second electrode is disposed distally of the distal set of magnets.

21-24. (canceled)

25. A system for forming a fistula between two vessels comprising:
a first catheter according to claim 1; and
a handle attached to the proximal end of the first catheter, wherein the handle comprises a toggle switch, wherein the toggle switch is configured to switch between providing power to the first electrode or the second electrode.

26-28. (canceled)

29. The system of claim 25, further comprising a second catheter having a backstop.

30-35. (canceled)

36. A method of creating arterial bloodflow in a vein, using a catheter having a catheter body with a first electrode and a second electrode, the method comprising:
inserting the catheter into a vein through an access site;
advancing the catheter to a treatment site where a fistula is to be formed;
upon encountering a valve, supplying RF energy to the second electrode to destroy said valve;
upon arriving at a treatment site where the fistula is to be formed, supplying RF energy to the first electrode to form the fistula.
